(19) United States
(12) Patent Application Publication
(10) Pub. No.: US 2023/0181786 A1
(43) Pub. Date: Jun. 15, 2023

(54) ADJUSTABLE UV IRRADIATION APPARATUS AND METHOD

(71) Applicant: Steven H. Larsen, Hayden, ID (US)

(72) Inventor: Steven H. Larsen, Hayden, ID (US)

(21) Appl. No.: 17/548,532

(22) Filed: Dec. 11, 2021

(51) Int. Cl. A61L 9/20; A61L 9/18

(52) U.S. Cl. CPC A61L 9/20; A61L 9/18; A61L 2209/12; A61L 2209/16

(57) ABSTRACT

An apparatus and method of decreasing airborne pathogens is provided. The apparatus is an array of UV radiating elements. The array uses two or more different types of UV radiators and is adaptable for positioning within an HVAC system for treating circulating air. The apparatus is also adaptable for use in other environments and may be mounted on a mobile motorized base for transportation within a structure in accordance with a predetermined, variable route. The method involves positioning the apparatus in an environment to be treated and testing radiation pattern and intensity, which can be adjusted for optimal treatment of the ambient air.

Applicant claims all the benefits of priority from U.S. Provisional Application 63124504 filed Dec. 11, 2020, the contents of which are hereby incorporated by reference in its entirety.

### FIELD OF THE INVENTION

The present invention relates generally to pathogen control. More particularly, the invention relates to an apparatus and method for reducing pathogens in various environments using UV radiation.

### Background of the Invention

Various methods for treating airborne pathogens such as bacteria and viruses have been known for years. Enhanced ventilation, anti-viral spray disinfectants, and UV-C radiation are three well known methods. One of the most effective methods of dealing with bacteria, viruses, and other airborne pathogens is to use UV-C radiation.

Traditionally UV-C is created through the mercury arc lamps and through gas discharge tubes. These sources produce a complex spectrum of electromagnetic radiation and some of it is in the UV-C region and a smaller part of this UV-C is exactly in the region that is of interest in killing pathogens.

The use of UV radiation in an HVAC system to treat airborne pathogens is known. The radiating element is typically positioned in the ducts or at the furnace air intake of the system. These systems are somewhat effective but suffer from the drawback that the UV radiation intensity is variable, that is not evenly distributed, so that not all the airflow passing by the radiating element is properly treated. Also, these systems are not “one size fits all” in that the exact positioning and intensity of the radiation needs to be adjusted in accordance with the physical parameters of the installation environment.

Another method of pathogen control is filtration with a forced air HVAC unit. The filter is placed over the air intake where it collects pathogens before they are recirculated into the ambient environment. The problem with filters is that pathogens collect on or in the filter so that over time the filter itself is a source of pathogens.

The use of antibacterial/antiviral sprays can be very effective but is costly as it requires many treatments over the course of a day. The sprays contain aerosols which can be allergenic to some people. Thus, sprays are better used to treat surfaces as opposed to the ambient air.

### SUMMARY OF THE INVENTION

An apparatus and method of decreasing airborne pathogens is provided. The apparatus is an array of UV radiating elements. The array uses two or more different types of UV radiators and is adaptable for positioning within an HVAC system for treating circulating air. The apparatus is also adaptable for use in other environments and may be mounted on a mobile motorized base for transportation within a structure in accordance with a predetermined, variable route. The method involves positioning the apparatus in an environment to be treated and testing radiation pattern and intensity, which can be adjusted for optimal treatment of the ambient air.

It is a major object of the invention to provide an apparatus and method for treating airborne pathogens.

It is another object of the invention to provide an apparatus and method for treating airborne pathogens which uses UV radiation.

It is another object of the invention to provide an apparatus and method for treating airborne pathogens that can be used within an HVAC system.

It is another object of the invention to provide an apparatus and method for treating airborne pathogens which uses an array of UV radiators.

It is another object of the invention to provide an apparatus and method for treating airborne pathogens where the apparatus is positioned on a mobile platform.

It is another object of the invention to provide an apparatus and method for treating airborne pathogens where the apparatus is positioned in accordance with measured parameters in the placement environment.

### BRIEF DESCRIPTION OF THE DRAWINGS

FIG. 1 shows a perspective view of the inventive apparatus.

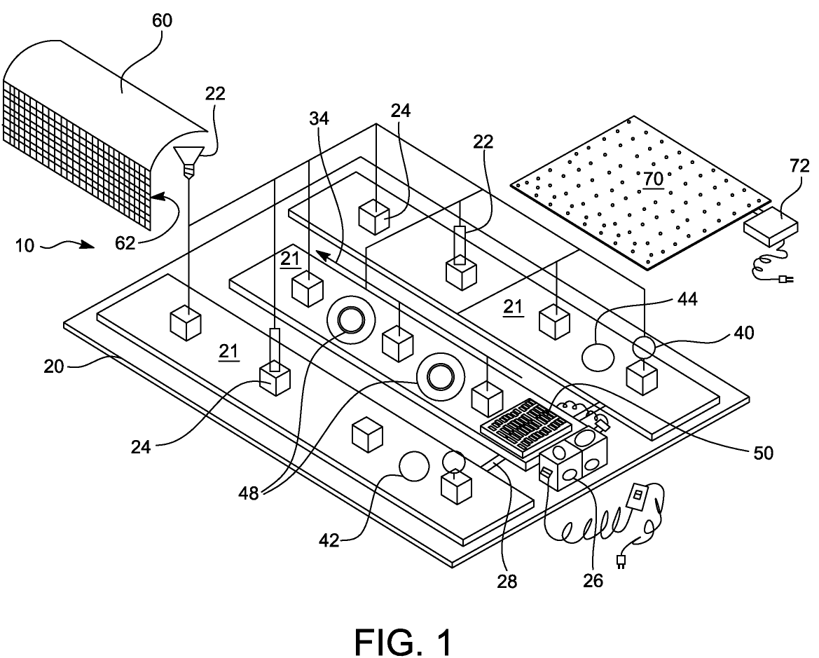

FIG. 2 shows a perspective view of the inventive apparatus modified for an automotive environment.

FIG. 3 shows a perspective view of an alternative embodiment of the inventive apparatus.

FIG. 4 shows a perspective view of another alternative embodiment of the inventive apparatus.

### DETAILED DESCRIPTION

The present invention is directed to an apparatus and method for treating airborne pathogens. The inventive apparatus, generally indicated by the numeral 10, is essentially a UV radiating device which is adaptable to different environments to allow for a wide variety of placement options. There are two alternative embodiments where the apparatus is configured for positioning in specific environments, but the basic operation is the same as will be explained in more detail below.

The apparatus 10 includes a mounting board or base 20 which is essentially a planar member which may be made from wood or any rigid non-conducting material. The base 20 is sufficiently large to accommodate at least 2 or 3 rows 21 of spaced UV radiating elements 22, but more or fewer rows 21 may be used depending upon the installation environment. At least two of the rows 21 include heat lamps 40 which can generate sufficient heat to kill pathogens. Each element 22 has a receptacle 24, which may be a plug in or threaded receptacle. Receptacles 24 are securely positioned on the board 20 in the well known manner with the attendant wiring. A GFI receptacle 26 is affixed to the board 20 and connected to a source of AC power. Lead wires 28 from the receptacle 26 are used to supply power to the rows 21, which may be light fixtures with bulb receptacles linearly arranged such as those on a vanity lighting bar commonly used in bathrooms and dressing rooms. The rows 21 may be removably secured to the board 20 by Velcro® or similar type fastener.

As is known in the art, UV-C bulbs come in a range of sizes and shapes which produce different radiation patterns and intensities. Also, the bulbs can be on constantly or pulsed. In accordance with one aspect of the invention, the shape, radiation pattern, intensity, and pulse rate (if any) of the UV-C radiators (bulbs 22) is adjusted based on the placement environment. Thus the invention overcomes the known difficulties of uneven air treatment of prior art systems, adjusting for environmental specifics such as the contours of the internal duct work and the shadows produced thereby. For example, if situated in an HVAC system environment a user will choose a variety of bulbs 22 initially, energize the apparatus 10, and measure the light/radiation dispersion, and adjust the placement of the apparatus 10, the type, and the positioning of bulbs 22 until an optimum dispersion of UV-C radiation is reached. Typically, this means there is no portion of the intake duct work in the immediate placement area that is not illuminated due to, e.g., shadows, and that the apparatus 10 is aligned with the short side 32 facing the direction of the airflow as shown by arrow 34. Also, in an ideal situation, the air is sampled to determine the effective kill rate of the pathogens, and the positioning, type of UV radiators, etc. is adjusted to optimize.

For the most effective treatment of pathogens UV-C radiation in the range of 230 to 280 nm is optimal. The bulbs 22 may have different frequencies within the 230 to 280 nm range. In addition to UV-C radiation, heat may be used to kill pathogens with the apparatus of the invention. To that end, a pair of heat lamps 40 are positioned proximate short side 32, the lamps spaced to more uniformly apply heat to the treatment area. A temperature gauge 42 and hygrometer 44 are used to measure temperature and humidity respectively, and these measurements may allow the user to determine the effectiveness of the apparatus 10.

The center row 21 includes at least two center mounted high intensity UV floodlamps 48, as well as an array of LEDs 50. The array 50 can be pulsed at a desired rate, which rate may be adjusted as described above. The array 50 is positioned proximate the short side 32 so that radiation therefrom pre-treats the incoming air before it passes through radiation from the other elements 22. The floodlamps 48 should be at least 100 watts each to ensure a minimum radiation of 300 watts regardless of the intensity of the other bulbs 22.

A partial enclosure 60 may be used with the apparatus, the enclosure having reflective material 62 such as aluminum positioned thereon. The enclosure 60 increases the effective UV-C radiation while being open ended to allow air flow. If placed within a duct, the reflective material 62 should be placed on the interior surfaces of the duct so that no air can pass underneath it, which, if it occurs would result in the air not being treated at all.

An optional LED array 70 may be attached to and suspended above the base 20. A controller 72 for adjusting the intensity and frequency of the illumination, as well as the temperature of the heat lamps 40 is electrically connected to the apparatus 10.

In operation, the apparatus 10 may be positioned within an HVAC system behind an opaque filter, or otherwise positioned so that none of the radiation escapes into a living space. Controller 72 is configured to sense when the HVAC system is on so that the apparatus 10 is not operational when there is no airflow to conserve power. Once positioned, the apparatus 10 will initiate when the HVAC system cycles on and airflow commences. The apparatus 10 will remain powered until the HVAC system cycles off.

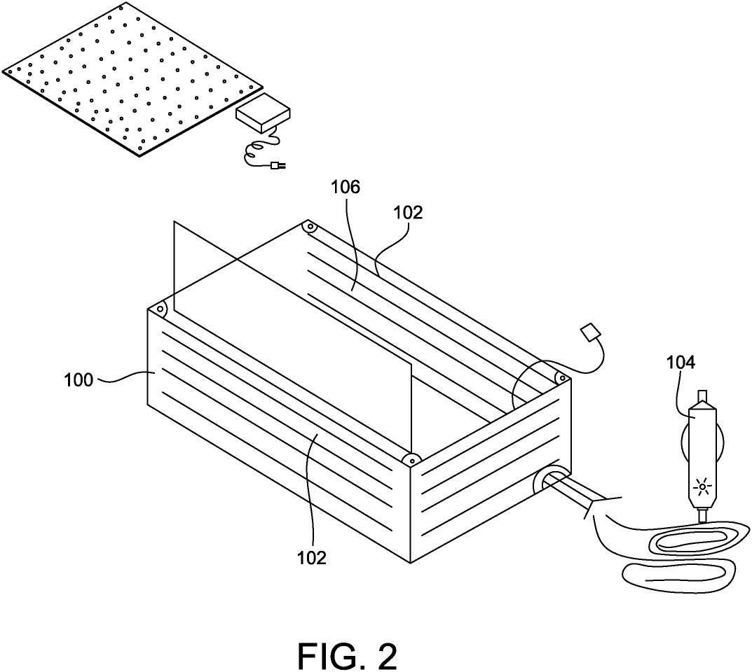

Referring now to FIG. 2 an alternative embodiment of the apparatus is shown. The modification includes a light tight enclosure 100 in which the apparatus 10 may be positioned. The enclosure 100 includes slits 102 having overlapping folds so as to allow airflow but prevent any radiation from escaping. The enclosure would be used in an automotive environment, and includes a standard automotive 12 volt auxiliary connector 104 for power. A top cover 106 can be removably positioned to make the enclosure light tight. The enclosure 200 with the apparatus 10 contained therein can be mounted on a mobile robotic device and can move within a structure as a mobile disinfection apparatus. The thus mobilized enclosure 200 may move within the structure in accordance with a predetermined pattern, or may be sent to rooms depending upon the occupancy rate so as to treat areas with “new” pathogens created by the occupants.

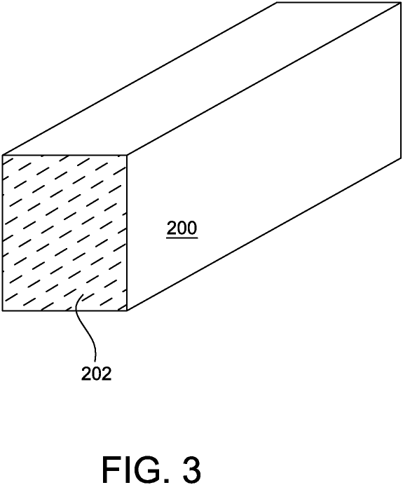

Referring now to FIG. 3 a series of LED strips 200 positioned inside an air duct 202 is shown. This may be used in the HVAC environment and is preferably positioned immediately after the apparatus with respect to the direction of airflow.

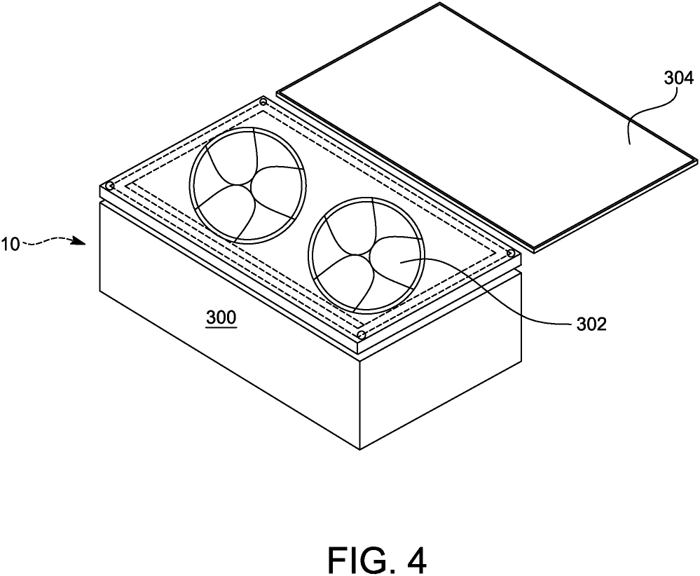

Referring to FIG. 4 another enclosure 300 for the apparatus 10 is shown. The enclosure 300 is light tight and includes fans 302 for circulating air. An opaque cover 304 is removably positioned on the enclosure 300, the cover formed of filtration material. The enclosure allows the apparatus 10 to be used in a home or office.

I claim:

1. An apparatus for treating airborne pathogens using radiation comprising:
a base, said base having at least two rows of radiating elements positioned thereon;
an array of pulsating LEDs positioned on said base;
at least two of said radiating elements being floodlamps; and,
at least two of heat lamps;
wherein intensity and frequency of said radiation is optimized for a given environment.

2. The apparatus of claim 1 wherein said at least two radiating elements radiate at different frequencies.

3. The apparatus of claim 1 wherein said radiation is UV-C radiation.

4. The apparatus of claim 3 wherein said UV-C radiation frequency is between 230 and 280 nm.

5. The apparatus of claim 1 including a reflecting member.
